(19) United States
(12) Patent Application Publication
(10) Pub. No.: US 2025/0161212 A1
(43) Pub. Date: May 22, 2025

(54) LIQUID PHARMACEUTICAL FORMULATION OF OMEPRAZOLE OR ESOMEPRAZOLE

(71) Applicant: ALKALOID AD SKOPJE, Skopje (MK)

(72) Inventors: Nadica VANOVA NAKJINOVA, Skopje (MK); Vlado PETRUSEVSKI, Skopje (MK); Ana JANEVSKA, Skopje (MK); Andrew WILLIS, Usk Monmouthshire (GB)

(21) Appl. No.: 18/834,984

(22) PCT Filed: Mar. 16, 2023
(86) PCT No.: PCT/EP2023/056779

§ 371 Date: Jul. 31, 2024

(30) Foreign Application Priority Data
Mar. 18, 2022 (GB) 2203764.2

(51) Int. Cl. A61K 9/107; A61K 31/4439; A61K 47/02; A61K 47/10; A61K 47/14; A61K 47/24; A61K 47/26; A61K 47/32; A61K 47/34; A61K 47/38; A61K 47/44

(52) U.S. Cl. CPC A61K 9/107; A61K 31/4439; A61K 47/02; A61K 47/10; A61K 47/14; A61K 47/24; A61K 47/26; A61K 47/32; A61K 47/34; A61K 47/38; A61K 47/44

(57) ABSTRACT

The present invention relates to a novel two-component formulation of an active ingredient, especially a proton pump inhibitor. The two-component formulation is stable for extended periods on storage, and the components are combined to give a formulation that is suitable for oral administration, having good palatability and efficacy.

### TECHNICAL FIELD

The present invention is directed to a two-part pharmaceutical formulation comprising a substituted benzimidazole, more specifically esomeprazole. The present invention also relates to preparations of such pharmaceutical formulations.

### BACKGROUND OF THE INVENTION

Proton pump inhibitors are a class of medicaments whose main action is a pronounced and long-lasting reduction of stomach acid production. They have the general structure (I)

(I)

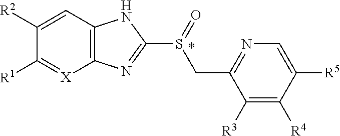

| Name | R¹ | R² | R³ | R⁴ | R⁵ | X |
|---|---|---|---|---|---|---|
| Timoprazole | H | H | H | H | H | CH |
| Omeprazole | OCH₃ | H | CH₃ | OCH₃ | CH₃ | CH |
| Lansoprazole | H | H | CH₃ | OCH₂CF₃ | H | CH |
| Pantoprazole | CF₂HO | H | OCH₃ | OCH₃ | H | CH |
| Rabeprazole | H | H | CH₃ | O(CH₂)₃OCH₃ | H | CH |
| Tenatoprazole | OCH₃ | H | CH₃ | OCH₃ | CH₃ | N |
| Picoprazole | CH₃ | CO₂CH₃ | CH₃ | H | H | CH |

Additionally, certain of the compounds shown in the table are used as single enantiomers (at the chiral centre indicated * in formula (I)). Esomeprazole is a single enantiomer of omeprazole. Dexlansoprazole is a single enantiomer of lansoprazole.

Omeprazole is a substituted benzimidazole, 6-methoxy-2-[[(4-methoxy-3, 5-dimethyl-2-pyridinyl) methyl]sulfinyl]-1H-benzimidazole that reduces gastric acid production by irreversibly inhibiting the H⁺/K⁺ ATPase (proton pump) at the secretory surface of the gastric parietal cells. The formula of omeprazole is

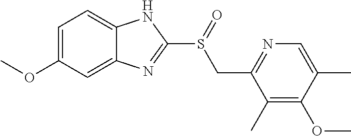

The proton pump is directly responsible for secreting H⁺ ions into the lumen of the stomach. By inhibiting the proton pump, omeprazole regulates the final step of hydrogen ion production in the pathway for gastric acid secretion in the gastrointestinal tracts of mammals.

Omeprazole and other proton pump inhibitors (PPIs) are used in the treatment of a number of conditions which require reduction in acid production by proton pump inhibition, such as gastritis, gastroesophageal reflux disease (GERD), dyspepsia, peptic ulcer disease, laryngopharyngeal reflux, gastric and duodenum ulceration and Zollinger-Ellison syndrome.

Typically, omeprazole is formulated as tablets or capsules for oral administration. However, such formulations present problems for patients who may be unable and/or unwilling to swallow capsules or tablets. This form is also undesirable for paediatric use.

A liquid formulation of omeprazole for oral administration has proven to be problematic due to the unstable nature of the active ingredient. Omeprazole is an acid labile compound and therefore, rapidly degrades in acidic conditions such as the environment found in the stomach. Omeprazole is known to degrade with a half-life of less than 10 minutes in an environment having a pH of below 4.0; at pH 6.5, the half-life is 18 hours and at pH 11, about 300 days.

An alternative to the above formulations is the provision of omeprazole as a solid suspension, in which granules of omeprazole are mixed with a suitable carrier. The suspension needs to be prepared immediately before use and it needs a strict control in the way it is prepared. As a result, there is a chance that the medicine may be rendered ineffective if not prepared correctly. Some patients also find the suspension very unpleasant to consume. Since the shelf-life is very short for omeprazole formulated as a suspension, it is also difficult to batch manufacture these.

One solution is to provide a liquid suspension of omeprazole which does not require controlled preparation. However, such suspensions are required to be of high viscosity to ensure that the microgranules of omeprazole are fully suspended in solution. This high viscosity makes it difficult to measure the correct dosage. Also, such formulations provide slow release of omeprazole and hence, they are limited in use.

It is therefore desirable to provide a stable pharmaceutical formulation comprising omeprazole or other PPIs which also addresses the problems identified above.

The present invention is related to a pharmaceutical formulation comprising omeprazole, esomeprazole, or other proton pump inhibitors, or a mixture thereof, which provides cost effective means for the treatment of the aforementioned conditions and is convenient to prepare. It also provides alternatives to patients unable and/or unwilling to ingest capsules. The present formulation is stable, easily dissolvable and it allows immediate release and rapid absorption of the therapeutic agent without it being degraded by the stomach acid.

US 2009/048322 relates to a drug delivery system, comprising a benzimidazole derivative, an oil, a surfactant, a cosurfactant and a dipolar aprotic solvent in a microemulsion formulation. The formulations appear to be adapted for pulmonary delivery.
WO 2003/086392 A2 relates to orally deliverable finely divided self-emulsifiable pharmaceutical compositions that comprise a drug of low water solubility i.e., a cyclooxygenase-2 inhibitory drug, more particularly to such compositions where the drug is in dissolved form, and to processes for preparing such compositions.
CA 2934538 A1 relates methods of preparing a medical solution comprising a therapeutic agent. It further relates to dry drug compositions (e.g., dry powder compositions) that can be dissolved and/or reconstituted rapidly for delivery to a patient.
The present invention addresses problems associated with the prior art.

### SUMMARY OF THE INVENTION

According to a first aspect, the invention provides a pharmaceutical formulation comprising
a first composition being a non-aqueous liquid comprising a substituted benzimidazole and a polar aprotic solvent; and
a second composition being an aqueous liquid comprising a base; wherein the first and the second compositions are configured to be combined before use to provide a combined liquid medicament having a pH in the range of between 7.5 to 9.5.

According to another aspect, the pharmaceutical formulation of the present invention is provided for use as a medicament to treat a condition selected from the group consisting of gastritis, gastroesophageal reflux disease, dyspepsia, peptic ulcer disease, laryngopharyngeal reflux, gastric and duodenum ulceration or Zollinger-Ellison syndrome.

According to another aspect, there is provided a kit comprising a first compartment containing a first composition as defined herein; a second compartment containing a second composition as defined herein; wherein the first and second containers are adapted to allow fluid communication between them on actuation to form a combined liquid medicament as defined herein.

According to another aspect, there is provided a method of treatment of a patient in need thereof of a condition selected from the group consisting of gastritis, gastroesophageal reflux disease, dyspepsia, peptic ulcer disease, laryngopharyngeal reflux, gastric and duodenum ulceration or Zollinger-Ellison syndrome, comprising admixing a first composition as defined herein and a second composition as defined herein and administering the combined liquid medicament to the patient.

### BRIEF DESCRIPTION OF THE FIGURES

FIG. 1 shows a two-part container suitable for storing the pharmaceutical formulation of the invention.

### DETAILED DESCRIPTION OF THE INVENTION

In accordance with the present invention, there is provided a two-part pharmaceutical formulation comprising a first and a second composition wherein the first composition is a non-aqueous liquid comprising a substituted benzimidazole and a polar aprotic solvent, and the second composition is an aqueous liquid comprising a base, wherein the two compositions are combined to achieve a combined liquid medicament for oral use.

In a preferred embodiment, the substituted benzimidazole is selected from the group of proton pump inhibitors. The proton pump inhibitor is preferably one of those defined in Formula (I) above, including pharmaceutically acceptable salts, esters and enantiomeric mixtures thereof. More preferably the therapeutic agent is selected from the group consisting of omeprazole, esomeprazole, lansoprazole, dexlansoprazole, timoprazole, picoprazole, pantoprazole, rabeprazole, and tenatoprazole. These proton pump inhibitors may be used singularly or in combination with each other. These inhibitors provide lasting reduction of gastric acid production by blocking the hydrogen/potassium adenosine triphosphate enzyme system (H⁺/K⁺ ATPase) of the gastric parietal cells.

Preferably, the therapeutic agent is selected from omeprazole, esomeprazole, pantoprazole and pharmaceutically acceptable salts, enantiomers, alkaline salts, enantiomers, hydrates, or derivatives thereof. In a preferred embodiment, the therapeutic agent is omeprazole or a pharmaceutically acceptable salt. A pharmaceutically acceptable salt of the therapeutic agent may comprise alkali metal salts such as sodium, lithium and potassium salts. A pharmaceutically acceptable salt of the therapeutic agent may also comprise alkaline earth and transition metal salts such as calcium and magnesium salts. Particularly preferred are metal salts of esomeprazole, particularly esomeprazole sodium.

The concentration of the therapeutic agent present in the first composition is between about 10 mg/ml to about 200 mg/ml. Preferably, the amount of the therapeutic agent in the first composition may be between about 50 mg/ml to about 100 mg/ml. More preferably, the therapeutic agent is at a concentration of about 70 mg/ml. This therapeutic agent is found to be stable in the polar aprotic solvent at these concentrations.

Preferably, the first composition is present in an amount of from 1 to 5 mL, preferably about 2 mL. Preferably, the second composition is present in an amount of from 25 to 100 mL, preferably about 68 mL.

The therapeutic agent may be present in the first composition in any suitable form, such as in the form of a suspension, liquid, solution, powder, granules, dry powder, dry granules, or microgranules. The therapeutic agent may additionally be provided with a coating, such as, for example, an enteric coating which would be stable from degradation when in contact with an acidic medium, such as present in the stomach, but would break down rapidly at a higher pH, when in the gastrointestinal tract for absorption. In a preferred embodiment, the therapeutic agent is provided in the form of a liquid, preferably a solution.

As used herein, the term “polar aprotic solvent” refers to a solvent that lacks an acidic proton and is polar. Preferably, the polar aprotic solvent is at miscible with water. Preferably, the polar aprotic solvent is a sulphur-containing solvent. Most preferably, the polar aprotic solvent is dimethyl sulfoxide (DMSO).

The second composition of the present invention comprises a diluent and a base. The diluent may be any suitable liquid aqueous diluent, for instance, water, or a mixture of one or more solvents comprising water. Preferably, the diluent is water. The amount of the diluent depends on the final volume of the formulation required depending on the concentration of the therapeutic agent.

The second composition of the present invention may comprise one or more pharmaceutical excipients and/or adjuvants. These are preferably selected from one or more of the group consisting of viscosity building agents, dispersing agents, emulsifiers, stabilizers, preservatives, antifoam agents, flavouring agents, antioxidants, sequestering agents and tonicity adjusting agents.

### Viscosity Building Agents

Viscosity building agents (or thickening agents) serve to regulate the viscosity of the formulation and decrease the bitterness of the active pharmaceutical ingredient. The amount of the thickening agents depends on the viscosity of the final formulation and/or the therapeutic agent required. DV2T RV Viscometer is used for apparent viscosity measurement in the present invention. In a preferred embodiment, the viscosity of the final pharmaceutical formulation (combined solution) of the present invention may be from about 200 centipose to about 600 centipose. In a preferred embodiment, the viscosity of the final formulation is from about 300 centipose to about 400 centipose. The viscosity of the first composition may be from about 150 centipose to about 450 centipose or from about 200 centipose to about 300 centipose. The viscosity of the second composition may be from about 350 centipose to about 650 centipose, or from about 400 centipose to about 550 centipose. The thickening agent may be selected from carageenans, powdered cellulose, sodium alginate, methylcellulose, hydroxyl ethyl cellulose, hydroxyl propyl cellulose, carboxymethylcellulose, sodium carboxymethylcellulose/microcrystalline cellulose mixtures, ion-exchange cross-linked polyacrylic polymers, polysaccharides, starches, carbomers, or a mixture thereof. In a preferred embodiment, the thickening agent is a combination of polysaccharide and sodium carboxymethylcellulose. Xanthan gum is preferred.

### Emulsifiers

Emulsifiers serve to maintain the second composition in the form of a stable emulsion. Emulsifiers are preferably selected from polyglycerol esters, polysorbates, mono- and di-glycerides of fatty acid esters, phosphatidylcholine (lecithin), and polyoxyethylene derivatives of sorbitan fatty acid esters. Preferred emulsifiers have a hydrophilic-lipophilic balance (HLB) of greater than 8 in the case of water-based diluent/second composition, and lower than 8 in the case of emulsion diluent/second composition. Hydrophilic-lipophilic balance (HLB) may be assessed by the method of Davies Proceedings of the International Congress of Surface Activity, 1957, pp. 426-38, incorporated herein by reference. Preferred emulsifiers are selected from polyoxyethylene sorbitan fatty acid esters, polyoxyethylene castor oil derivatives, polyoxylglycerides of sorbitan monooleate, phosphatidylcholine, polyglyceryl-3 oleate and mixtures thereof.

### Stabilizers

Stabilizers are incorporated into the second composition to prevent aggregation. Stabilizers are preferably selected from PVP (povidone), PVA (polyvinyl alcohol), PEG (polyethylene glycol), HPMC (hypromellose), HPC (hydroxypropyl cellulose), HEC (hydroxyethyl cellulose), NaCMC (carboxymethylcellulose sodium), SD (docusate sodium), SLS (sodium lauryl sulphate), PEI (polyehtylene imine), TPGS (D-α-tocopheryl polyethylene glycol succinate), PEO (polyethylene oxide) and PPO (polypropylene oxide), amino acids (preferably Arginine, cysteine and/or glycine), poloxamers and mixtures thereof. Preferred stabilizers are selected from amino acids (preferably Arginine, cysteine and/or glycine), poloxamers and mixtures thereof. Poloxamer 407 is particularly preferred (BASF trade name Kolliphor P 407®).

### Preservatives

Preservatives preserve the second composition to prevent decomposition and microbial growth. The preservatives may be quaternary ammonium compounds (such as benzalkonium chloride (BAC), didecyldimethylammonium chloride (DDAC)), parabens, butylated hydroxytoluene, butylated hydroxyanisole, tert-butylhydroquinone EDTA, disodium EDTA or a salt thereof; sugar alcohol such as glycerol and sorbitol; polyoxyethylene 20 sorbitan monooleate such as polysorbate 80, polyoxyethylene 20 sorbitan monolaurate like polysorbate 20; disodium phosphate; or a mixture thereof.

Preferred preservatives are selected from quaternary ammonium compounds, parabens, butylated hydroxytoluene, butylated hydroxyanisole, tert-butylhydroquinone and mixtures thereof. The preservatives may be about 0.01% (w/v) to about 2% (w/v), more preferably, about 0.1% (w/v).

### Antifoam Agents

The anti-foaming agent prevents the formation of any bubbles or foaming. Suitable antifoaming agents may be selected from silicone based antifoaming agent such as, simethicone or its emulsion or suspension, and non-silicone anti-foaming agents like polypropylene based polyether dispersions, castor oil, fatty alcohol esters, glycerides and a mixture thereof. A preferred antifoam agent is simethicone. The antifoam agent is preferably present in an amount of from 0.05 to 0.5% (w/v).

### Flavouring Agents

The sweetening agents and flavouring agents are used to mask the bitter taste of the active pharmaceutical ingredient and thus, a formulation with good palatability and acceptability is provided. Suitable sweetening agents for use in the present invention are sugars, sugar alcohols, sodium saccharide, aspartame, acesulfame potassium, stevia, sodium cyclamate, propylene glycol, liquorice extract, glycyrrhizin, maple extract and locust bean extract. Suitable flavouring agents may be selected from taste-mask sweetness flavour, sucralose, menthol (mint), lemon, orange, peach, cinnamon, black current, cherry and chocolate. The sweetening agents and flavouring agents may be present in an amount from about 1.0% (w/v) to about 0.01% (w/v). More preferably, these agents may be present in an amount from about 0.25% (w/v), 0.2% (w/v), 0.15% (w/v), or 0.015% (w/v).

### Antioxidants

Suitable antioxidants for use in the second composition are selected from ascorbic acid derivatives, thiol derivatives, sulphites, sodium sulfates, synthetic hindered phenols like propyl gallate, butylated hydroxyanisole (BHA), butylated hydroxytoluene (BHT), natural hindered phenols like tocopherols, or a mixture thereof. The antioxidant lends stability to the therapeutic agent and prevents oxidative degradation. The amount of antioxidant in the composition may be from about 0.05% (w/v) to about 0.5% (w/v), preferably from about 0.1% (w/v) to about 0.3% (w/v) of the first composition. However, it is preferred that no antioxidant is present.

### Tonicity Adjusting Agents

The purpose of the tonicity adjusting agent (osmotic agents) used in this invention is to give the combined formulation an osmolality in the range of from about 1000 to about 3000 mOsmol/kg. In a preferred embodiment, the osmolality may be from about 1000 to about 1500 mOsmol/kg or from about 1500 to about 2500 mOsmol/kg or from about 1500 to about 2200 mOsmol/kg. The presence of osmotic agents within this range ensures that the pharmaceutical formulation achieved has acceptable hypertonicity such that the formulation can be consumed in an undiluted form. The osmotic agents may be selected from dextrose, mannitol, sorbitol, glycerol, sodium chloride and sodium sulphate or a combination thereof.

In the context of this invention, the base serves to regulate the pH of the formulation. Thus, the base is capable of modifying the pH of the final (i.e. combined first and second composition) formulation to obtain a desired pH that prevents the therapeutic agent form being degraded by the acidic gastric fluids, and is suitable for oral administration. In a preferred embodiment, the pH of the final formulation (combined solution) is between 7.5 and 9.5. In a more preferred embodiment, the pH of the final formulation is between 8 and 9. In a more preferred embodiment, the pH of the combined solution is between pH 8.5 and 9. In a more preferred embodiment, the pH of the combined solution is about 9. The base may be selected from alkali metal or alkaline earth metal carbonates or bicarbonates, alkali metal or alkaline earth metal hydroxides, alkali metal or alkaline earth metal oxides, alkali metal or alkaline earth metal phosphates, citrates, acetates and a mixture thereof. Preferably, the base is potassium bicarbonate.

The base ensures that when the first and second compositions are mixed, the combined medicament brought back within the acceptable range, and hence the pH of the combined liquid medicament is consistent and stable on administration.

The therapeutic agent used in this invention is preferably sparingly soluble in water at pH 7. In some embodiments, the second composition comprises a co-solvents in this invention is to improve the solubility of the active pharmaceutical ingredient in the combined preparation. The solvents and co-solvents may be selected from glycerol, sorbitol, propylene glycol, alcohol, polyethylene glycols, or a mixture thereof. In a preferred embodiment, the solvents and co-solvents may be sugar alcohol. The solvents and co-solvents may be present in amount from about 5% (w/v) to about 20% (w/v), more preferably, from about 10% (w/v) to about 15% (w/v).

The function of the dispersing agents is to aid sparingly soluble components to dissolve properly. Suitable dispersing agents for use in this invention are glycerol, sorbitol, propylene glycol, polyethylene glycols, alcohol and combinations thereof.

Preferably, the first and the second composition are both provided in the form of a solution. In a more preferred embodiment, the combined liquid medicament is homogeneous.

The first composition and second composition are provided in volumes such that when combined, the volume of combined liquid medicament is acceptable for a several individual oral doses. Typically, the volume of combined liquid medicament is between 1 and 150 ml, preferably 20 to 100 ml, more preferably 50 to 90 ml, such as around 70 ml. Accordingly, the volumes of the first and second compositions are selected to provide such a total volume of combined liquid medicament. The volume of combined medicament is suitable to provide from 1 to 50 individual doses, such as between 5 to 20 individual doses. Individual doses comprise from 5 to 50 mg, preferably 10 to 40 mg. As will be apparent to the person skilled in the art, the clinician will measure out the appropriate dose of the combined liquid medicament and administer to the patient in need thereof.

In another embodiment the present invention provides a kit that comprises the first and second compositions, such that both the compositions are stored separately and are combined to form a combined liquid immediately prior to use. The resulting combined liquid medicament preferably has a pH of from 7.5 to 9.5 and this pH stability is maintained for at least 7 days.

The first and second compositions, kept separately, can be stored for at least 12 months and the active pharmaceutical agent maintains its activity for at least 12 months.

The kit may comprise a removable barrier between the two compositions. The removable barrier is configured to prevent contact between the two compositions whereby the removal of the removable barrier allows combining of the two compositions immediately before consumption to provide the combined solution. In a preferred embodiment, the barrier is a breakable membrane.

Optionally, the removable barrier may be reusable as long as it can separate the two compositions and be removed so as to allow mixing of the two compositions. For example, the removable barrier may be two separate containers for the two compositions having removable openings such as caps or lids. Alternatively, the removable barrier may have a feature of a single container comprising the two compositions separated by the removable barrier. Preferably, the removable barrier is made of plastic or glass. More preferably, the removable barrier is made of amber high-density polyethylene.

The pharmaceutical formulation of the present invention is provided for use in the treatment of a number of conditions such as gastritis, gastroesophageal reflux disease, dyspepsia, peptic ulcer disease, laryngopharyngeal reflux, gastric and duodenum ulceration, and Zollinger-Ellison syndrome whereby the condition is treated by proton pump inhibition. The pharmaceutical formulation of the present invention is suitable for adults and children.

In use, the clinician or patient admixes the first and second compositions to form a combined liquid, which is then directly orally administered to the patient.

The formulations and kits of the invention combine a long shelf-life with minimal degradation of the active substance, together with a convenient and palatable dosage form for administration.

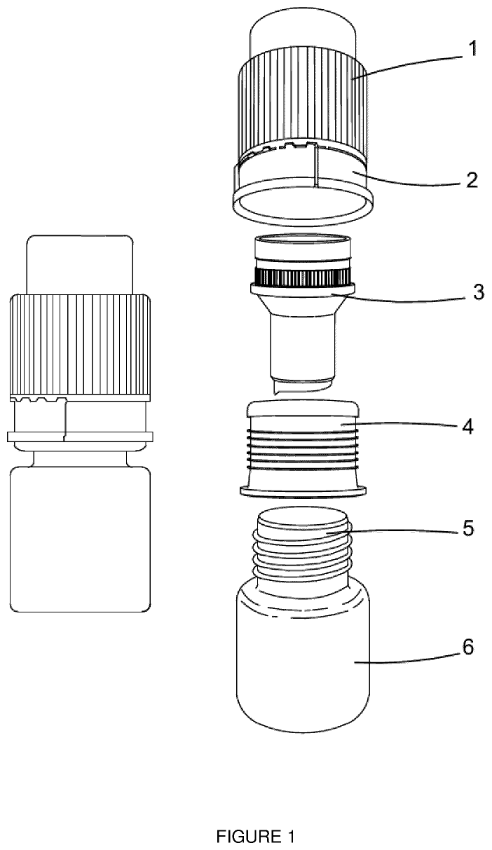

With reference to FIG. 1, a suitable dual—chamber multi dose pack for storage and mixture of the compositions of the invention comprises:

Cap with Tamper Evident Tear Band

Cap (1) over the first chamber comprising a means to exert pressure onto the plunger (3) so as to partially rupture the breakable polymeric membrane (4) of the plug and deliver the solution into the container (6) with the second liquid composition, diluent for the esomeprazole solution in amount of 68 mL.

### Tamper Evident Tear Band (2)

The tamper evident tear band (2) ensures the overall integrity of the product until the time of the administration.

A first chamber composed comprising:

### Plunger—First Container (3)

Plunger (3) adapted to fit into a plug (4) having a top flat surface, containing a first composition esomeprazole oral solution in amount of 2 ml.

### Plug (4)

The plug (4) with breakable polymeric membrane, adapted to fit into the opening bottle liner (5) from the lower end into a cap (1) from the upper end. Second chamber comprising:

### Opening Bottle Liner (5)

### Container—Second Container (6)

A second chamber in the form of a container (6) provided with an opening bottle liner (5) at an upper end, comprising liquid diluent for the esomeprazole oral solution in amount of 68 ml. In container (6) the two liquid compositions are mixed at the time of the administration.

### EXAMPLES

The following examples further illustrate the invention:

### Example 1 (Emulsion) 20 mg/10 ml, 70 ml

### First Chamber:

Esomeprazole 0.140 g or 0.149 g as sodium salt in 2 ml DMSO solvent

### Second Chamber (68 mL)

| Constituent | Function | Amount (w/v) |
|---|---|---|
| Potassium hydrogen | Base, buffering agent | 2.8% |
| carbonate | | |
| Sodium carbonate | Base, buffering agent | 0.7% |
| Sucralose | Sweetener | 0.3% |
| Carboxymethyl cellulose | Stabilizer, viscosity | 0.4% |
| sodium | modifying agent | |
| Polysorbate 80 | Emulsifier, surfactant | 3% |
| Simethicone emulsion | Antifoaming agent | 0.08% |
| Water purified | Hydrophilic phase | 25% |
| Phosphatidylcholine | Emulsifier, surfactant | 5% |
| Methylparaben | Preservative | 0.2% |
| Butylated hydroxytoluene | Antioxidant | 0.01% |
| Flavour lemon | Flavour | 0.12% |
| Flavour Mint | Flavour | 0.02% |
| Olive oil | Lipophilic phase | Balance |

### Method:

The hydrophilic and hydrophobic phases were prepared separately with heating up to 65°.

The potassium hydrogen carbonate, sodium carbonate, sucralose, polysorbate 80, and simethicone emulsion were dissolved in water, and heated to 65° C. (hydrophilic phase).

The phosphatidylcholine, methylparaben, and butylated hydroxytoluene were dissolved in heated olive oil to 75° C. (lipophilic phase), Carboxymethylcellulose sodium is dispersed afterwards.

The hydrophilic phase was dispersed in the lipophilic phase under high speed homogenization at 1500-4000 rpm for 15 minutes.

Mixing with cooling was conducted for a minimum of 1 hour, at 80 rpm using a propeller mixer and 0.3 bar negative pressure.

At 30° C., the flavours were added and mixed for additional 1 hour. A stable emulsion was obtained.

The two compositions were stored in a dual chamber system, where the API in DMSO was contained in the first chamber (made of HDPE/PE/PP) and 68 ml of diluent in the second chamber. The second chamber was made of HDPE or PET plastic with a volume of 100 ml.

Immediately before first use, the two compositions were combined, and shaken. The appropriate volume of combined formulation is measured out with e.g. a syringe or dosing spoon, and administered to a patient in need thereof.

### Example 2 (Emulsion) 20 mg/10 ml, 70 ml

### First Chamber:

Esomeprazole 0.140 g or 0.1499 g used as sodium salt in 2 ml DMSO solvent

### Second Chamber: (68 mL)

| Constituent | Function | Amount (w/v) |
|---|---|---|
| Potassium hydrogen | Base, buffering agent | 2.8% |
| carbonate | | |
| Sodium carbonate | Base, buffering agent | 0.7% |
| Sucralose | Sweetener | 0.3% |
| Carboxymethyl cellulose | Stabilizer, viscosity | 0.4% |
| sodium | modifying agent | |
| PEG 40 hydrogenated | Emulsifier, surfactant | 2% |
| castor oil | | |
| Simethicone emulsion | Antifoaming agent | 0.08% |
| Water purified | Hydrophilic phase | 25% |
| Phosphatidylcholine | Emulsifier, surfactant | 5% |
| Polyglyceryl-3 oleate | Emulsifier, surfactant | 1.5% |
| Methylparaben | Preservative | 0.2% |
| Butylated hydroxytoluene | Antioxidant | 0.01% |
| Flavour lemon | Flavour | 0.12% |
| Flavour Mint | Flavour | 0.02% |
| Olive oil | Lipophilic phase | Balance |

The emulsion was prepared by a similar method to Example 1—potassium hydrogen carbonate, sodium carbonate, sucralose, carboxymethyl cellulose sodium, PEG 40 hydrogenated castor oil, and simethicone emulsion formed the hydrophilic phase, and phosphatidylcholine, polyglyceryl-3 oleate, methylparaben and butylated hydroxytoluene formed the lipophilic phase.

### Example 3 (Water-Based Solution) 20 mg/10 ml, 70 ml

### First Chamber:

Esomeprazole 0.140 g or 0.149 g used as sodium salt in 2 ml DMSO solvent

### Second Chamber: (68 mL)

| Constituent | Function | Amount (w/v) |
|---|---|---|
| Potassium hydrogen | Base, buffering agent | 4% |
| carbonate | | |
| Sodium carbonate | Base, buffering agent | 1% |
| Sucralose | Sweetener | 0.4% |
| Polyacrylic acid polymer | Stabilizer, viscosity | 0.25% |
| | modifying agent | |
| Carboxymethyl cellulose | Stabilizer, viscosity | 1% |
| sodium | modifying agent | |
| Hydroxypropyl beta | Taste masking agent | 0.5% |
| cyclodextrin | | |
| Maltodextrin | Taste masking agent | 2% |
| Sodium chloride | Taste masking agent | 0.5% |
| Polysorbate 80 | Wetting agent, surfactant | 0.25% |
| Simethicone emulsion | Antifoaming agent | 0.08% |
| Flavour lemon | Flavour | 0.12% |
| Flavour Mint | Flavour | 0.02% |
| Benzalkonium chloride | Preservative | 0.02% |
| Water purified | Solvent | Balance |
| Sodium hydroxide | Base | q.s. pH 9.0-9.5 |

### Method:

The potassium hydrogen carbonate and sodium carbonate salts were dissolved ⅔ of the water. Subsequently, the benzalkonium chloride was dissolved with a mixing time of 15 minutes in a propeller mixer at 80 rpm.

Sodium chloride, maltodextrin and simethicone emulsion were dissolved sequentially with in-between mixing times of 15 minutes in a propeller mixer at 80 rpm.

After complete dissolution, cyclodextrin and sweetener were dissolved sequentially with in-between mixing times of 15 minutes. The polymer was added at a higher stirrer speed (at least 100 rpm) in a propeller mixer in 3 portions.

The polymer was hydrated for 1 hour, and after full hydration the remaining flavours were added and mixed for an additional 30 minutes with propeller mixer operating at a minimum of 100 rpm.

The pH adjusted to 9.0-9.5 with 1 M NaOH solution, and the volume adjusted to 68 mL with water.

The two compositions were stored in a dual chamber system, where the API in DMSO was contained in the first chamber (made of HDPE/PE/PP) and 68 ml of diluent in the second chamber (aqueous solution). The second chamber was made of HDPE plastic, PET or (preferably) amber glass with volume of 120-130 ml.

Immediately before first use, the two compositions were combined, and shaken. The appropriate volume of combined formulation is measured out with e.g. a syringe or dosing cup and administered to a patient in need thereof.

### Example 4 (Water-Based Solution) 20 mg/10 ml, 70 ml

### First Chamber:

Esomeprazole 0.140 g or 0.149 g used as sodium salt in 2 ml DMSO solvent

### Second Chamber: (68 mL)

| Constituent | Function | Amount (w/v) |
|---|---|---|
| Potassium hydrogen | Base, buffering agent | 4% |
| carbonate | | |
| Sodium carbonate | Base, buffering agent | 1% |
| Sucralose | Sweetener | 0.4% |
| Polyacrylic acid polymer | Stabilizer, viscosity | 0.25% |
| | modifying agent | |
| Polyvinyl alcohol | Stabilizer, viscosity | 0.5% |
| | modifying agent | |
| Hydroxypropyl beta | Taste masking agent | 0.5% |
| cyclodextrin | | |
| Maltodextrin | Taste masking agent | 2% |
| Sodium chloride | Taste masking agent | 0.5% |
| Polysorbate 80 | Wetting agent, surfactant | 0.25% |
| Phosphatidylcholine | Wetting agent, surfactant | 0.25% |
| Simethicone emulsion | Antifoaming agent | 0.08% |
| Flavour lemon | Flavour | 0.12% |
| Flavour Mint | Flavour | 0.02% |
| Benzalkonium chloride | Preservative | 0.02% |
| Water purified | Solvent | Balance |
| Sodium hydroxide | Base | q.s. pH 9.0-9.5 |

The solution was made by a similar method to Example 4.

### Example 5 (Water Based Solution) 20 mg/10 ml, 70 ml

### First Chamber:

Esomeprazole 0.140 g or 0.1499 g used as sodium salt in 2 ml DMSO solvent

### Second Chamber: (68 ml)

| Constituent | Function | Amount (w/v) |
|---|---|---|
| Potassium hydrogen | Base, buffering agent | 2.8% |
| carbonate | | |
| Sodium carbonate | Base, buffering agent | 0.7% |
| Sucralose | Sweetener | 0.4% |
| Carboxymethylcellulose | Stabilizer, viscosity | 1% |
| sodium | modifying agent | |
| Polyvinyl alcohol | Stabilizer, viscosity | 0.5% |
| | modifying agent | |
| Glycerol | Co-solvent | 10% |
| Hydroxypropyl beta | Taste masking agent | 0.5% |
| cyclodextrin | | |
| Maltodextrin | Taste masking agent | 2% |
| Sodium chloride | Taste masking agent | 0.5% |
| Polysorbate 80 | Wetting agent, | 0.2% |
| | surfactant | |
| Simethicone emulsion | Antifoaming agent | 0.08% |
| Flavour lemon | Flavour | 0.12% |
| Flavour Mint | Flavour | 0.02% |
| Benzalkonium chloride | Preservative | 0.02% |
| Water purified | Solvent | Balance |
| Sodium hydroxide | Base | q.s. pH 9.0-9.5 |

The solution was made by a similar method to Example 4.

### Example 6 (Water Based Solution) 20 mg/10 ml, 70 ml

### First Chamber:

Esomeprazole 0.140 g or 0.1499 g used as sodium salt in 2 ml DMSO solvent

### Second Chamber: (68 ml)

| Constituent | Function | Amount mg/ml | |
|---|---|---|---|
| Potassium hydrogen | pH modifier | 28.82 | mg |
| carbonate¹ | | | |
| Sodium carbonate¹ | pH modifier | 7.21 | mg |
| Polyoxyl 40 Hydrogenated | wetting agent | 2.50 | mg |
| Castor Oil¹ | | | |
| Poloxamer 407¹ | solubilising agent, | 1.03 | mg |
| | stabilisation agent | | |
| Sucralose¹ | sweetening agent | 4.12 | mg |
| Emulsion simethicone | antifoaming agent | 0.44 | mg |
| 30%² | | | |
| Sodium alginate¹ | viscosity building agent, | 7.20 | mg |
| | taste masking agent | | |
| Edetate disodium¹ | sequestering agent, | 0.44 | mg |
| | preservative synergistic | | |
| | agent | | |
| Maltodextrin¹ | stabilisation agent, taste | 14.41 | mg |
| | masking agent | | |
| Hydrohypropyl beta | taste masking agent | 5.15 | mg |
| cyclodextrine¹ | | | |
| Methyl paraben/Propyl | antimicrobial | 2.00-6.62 | mg |
| paraben¹ | preservative | 0.15-0.46 | mg |
| Or Benzalconium chloride¹ | | 10.00-22.06 | mg |
| Or Benzyl alcohol¹ | | | |
| Flavour lemon³ | flavouring agent | 1.20 | mg |
| Flavour grape³ | flavouring agent | 1.50 | mg |
| Taste masking composition | flavouring agent | 1.00 | mg |

| | | |
|---|---|---|
| Water, purified¹ | solvent | ad 1.00 ml |

¹Ph. Eur. current edition of the European Pharmacopoeia;
²USP current edition of United states pharmacopoeia
³Regulation (EC) N° 1334/2008

### Example 7 (Oil in Water Oral Emulsion) 20 mg/10 ml, 70 ml

### First Chamber:

Esomeprazole 0.140 g or 0.149 g used as sodium salt in 2 ml DMSO solvent

### Second Chamber: (68 mL)

| | | Amount | |
|---|---|---|---|
| Constituent | Function | mg/ml | |
| Potassium hydrogen | pH modifier | 28.82 | mg |
| carbonate¹ | | | |
| Sodium carbonate¹ | pH modifier | 7.21 | mg |
| Xanthan gum¹ | viscosity building agent | 4.12 | mg |
| Polyoxyl 40 Hydrogenated | emulsifier | 10.29 | mg |
| Castor Oil¹ | | | |
| Poloxamer 407¹ | solubilisation agent, | 10.29 | mg |
| | stabilisation agent | | |
| Sucralose¹ | sweetening agent | 4.12 | mg |
| Emulsion simethicone | antifoaming agent | 0.44 | mg |
| 30%² | | | |
| Phosphatidylcholine | emulsifier | 30.88 | mg |
| (Lecithin)² | | | |
| Glycerol monostearate | structuring agent | 57.73 | mg |
| Or cetostearyl alcohol¹ | | 82.21 | mg |
| Medium chain triglyceride¹ | oil (dispersed) phase | 150.00 | mg |
| Methyl paraben/Propyl | antimicrobial | 2.00-6.62 | mg |
| paraben¹ | preservative | 0.15-0.46 | mg |
| Or Benzalconium chloride¹ | | 10.00-22.06 | mg |
| Or Benzyl alcohol¹ | | | |
| Flavour lemon³ | flavouring agent | 1.20 | mg |
| Flavour grape³ | flavouring agent | 1.50 | mg |
| Taste masking | flavouring agent | 1.00 | mg |
| composition | | | |

| | | |
|---|---|---|
| Water, purified¹ | continuous phase | ad 1.00 ml |

¹Ph. Eur. current edition of the European Pharmacopoeia;
²USP current edition of United states pharmacopoeia
³Regulation (EC) N° 1334/2008

### Example 8 (Oil in Water Oral Emulsion) 20 mg/10 ml, 70 ml

### First Chamber:

Esomeprazole 0.140 g or 0.1499 g used as sodium salt in 2 ml DMSO solvent

### Second Chamber: (68 mL)

| Constituent | Function | Amount mg/ml | |
|---|---|---|---|
| Potassium hydrogen | pH modifier | 28.82 | mg |
| carbonate¹ | | | |
| Sodium carbonate¹ | pH modifier | 7.21 | mg |
| Xanthan gum¹ | viscosity building agent | 4.12 | mg |
| Polysorbate 80 | emulsifier | 24.00 | mg |
| (Polyoxyethylene 20 | | | |
| sorbitan monooleate)¹ | | | |
| Span 80 (Sorbitan | emulsifier | 16.00 | mg |
| monooleate)¹ | | | |
| Glycerol monostearate | structuring agent | 30.60 | mg |
| Or Cetostearyl alcohol¹ | | 43.75 | mg |
| Poloxamer 407¹ | solubilising agent, | 8.00 | mg |
| | stabilisation agent | | |
| Sucralose¹ | sweetening agent | 4.12 | mg |
| Emulsion simethicone | antifoaming agent | 0.44 | mg |
| 30%² | | | |
| Olive oil, refined¹ | oil (dispersed) phase | 100.00 | mg |
| Methyl paraben¹/Propyl | antimicrobial | 2.00-6.62 | mg |
| paraben¹ | preservative | 0.15-0.46 | mg |
| Or Benzalconium chloride¹ | | 10.00-22.06 | mg |
| Or Benzyl alcohol¹ | | | |
| Flavour lemon³ | flavouring agent | 1.20 | mg |
| Flavour grape³ | flavouring agent | 1.50 | mg |
| Taste masking | flavouring agent | 1.00 | mg |
| composition | | | |

| | | |
|---|---|---|
| Water, purified¹ | continuous phase | ad 1.00 ml |

¹Ph. Eur. current edition of the European Pharmacopoeia;
²USP current edition of United states pharmacopoeia
³Regulation (EC) N° 1334/2008

### Example 9—Stability Study

Stability results for esomeprazole sodium dissolved in DMSO stored at 25° C.:

| Initial | 1.5 months | 5 months |
|---|---|---|
| Imp D = 0.03% | Imp D = 0.03% | RRT 0.57 = 0.02% |
| RRT 1.19 = 0.02% | RRT 1.23 = 0.03% | Imp D = 0.03% |
| RRT 1.23 = 0.03% | Total = 0.14% | RRT 1.14 = 0.02% |
| Total = 0.12% | | RRT 1.17 = 0.03% |
| | | RRT 1.23 = 0.02% |
| | | Total = 0.15% |

| | Initial | 1.5 months | 3 months | 5 months | 9 months | 13 months |
|---|---|---|---|---|---|---|
| Imp-s > | Imp D = | Imp D = | Imp D = | Imp D = | RRT 0.53 = | RRT 0.54 = |
| 0.02% | 0.03% | 0.03% | 0.03% | 0.03% | 0.03% | 0.04% |
| | RRT 1.23 = | RRT 1.23 = | RRT 1.23 = | RRT 1.17 = | Imp D = | RRT 0.90 = |
| | 0.03% | 0.03% | 0.03% | 0.03% | 0.03% | 0.04% |
| | | | | | RRT 1.23 = | Imp D = |
| | | | | | 0.03% | 0.03% |
| | | | | | RRT 1.31 = | RRT 1.23 = |
| | | | | | 0.03% | 0.03% |
| | | | | | | RRT 1.31 = |
| | | | | | | 0.04% |
| | | | | | | RRT 1.32 = |
| | | | | | | 0.03% |
| Total | 0.12% | 0.14% | 0.17% | 0.16% | 0.25% | 0.36% |
| impurities | | | | | | |

Key:
Imp D: omeprazole impurity D:

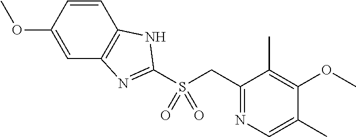

RRT: Relative retention time.

### Example 10—Palatability Study

The aqueous emulsion and the combined medicament of Example 2 was judged as palatable by a cohort of volunteers.

1. A pharmaceutical formulation comprising
a first composition being a non-aqueous liquid comprising a substituted benzimidazole selected from omeprazole, esomeprazole, and pharmaceutically acceptable salts thereof as a solution in dimethyl sulfoxide; and
a second composition being an aqueous liquid comprising a base;
wherein the first and the second compositions are configured to be combined before use to provide a combined liquid medicament having a pH in the range of between 7.5 to 9.5.

2. A pharmaceutical formulation according to claim 1 wherein the second composition comprises one or more pharmaceutical excipients and/or adjuvants, preferably selected from the group consisting of viscosity building agents, dispersing agents, emulsifiers, stabilizers, preservatives, antifoam agents, flavouring agents, antioxidants, sequestering agents and tonicity adjusting agents.

3. A pharmaceutical formulation according to claim 1, wherein the substituted benzimidazole has a concentration of between 0.5 mg/ml to 5 mg/ml after combining the first and the second compositions.

4. A pharmaceutical formulation according to claim 1 wherein the second composition is an emulsion or a solution.

5. A pharmaceutical formulation according to claim 4, wherein the second composition is a water in oil (w/o) emulsion further comprising an emulsifier system and a lipophilic phase.

6. A pharmaceutical formulation according to claim 5, wherein the second composition is an oil in water (o/w) emulsion further comprising an emulsifier system and a lipophilic phase.

7. A pharmaceutical formulation according to claim 5, wherein the emulsifier system comprises an emulsifier with HLB<8, preferably sorbitan monooleate, phosphatidylcholine, polyglyceryl-3 oleate or a combination thereof.

8. A pharmaceutical formulation according to claim 6, wherein the emulsifier system comprises an emulsifier with HLB>8, preferably polysorbate 80, polyoxyl 40, hydrogenated castor oil or a combination thereof.

9. A pharmaceutical formulation according to claim 5, wherein the lipophilic phase is selected from medium and long chain triglycerides, preferably olive oil or caprylic capric triglycerides.

10. A pharmaceutical formulation according to claim 5 wherein the lipophilic phase is present in an amount of below 20% w/w based on the weight of the second composition.

11. A pharmaceutical formulation according to claim 5 wherein the emulsion further comprises a waxy compound, preferably glycerol monostearate, cetostearyl alcohol, glycerol dibehenate, or a combination thereof.

12. A pharmaceutical formulation according to claim 1 wherein the base is selected from metal carbonates and metal bicarbonates or a combination thereof, preferably sodium carbonate, sodium or potassium bicarbonate or a combination thereof.

13. A pharmaceutical formulation according to claim 1 comprising a taste masking agent preferably selected from cyclodextrin, maltodextrin, and sodium chloride or a combination thereof.

14. A pharmaceutical formulation according to claim 1 comprising a viscosity building agent preferably selected from cellulose-based polymers, xanthan gum, sodium alginate and acrylic polymer or a combination thereof.

15. A pharmaceutical formulation according to claim 1 wherein the first and/or second composition comprises one or more additional components selected from the group of stabilizers, wetting agents, antifoaming agents, preservatives, antimicrobial agents, flavouring agents and sweeteners.

16. A pharmaceutical formulation according to claim 1 wherein the formulation is a multi-dose formulation.

17. A pharmaceutical formulation according to claim 1 wherein the formulation is stable at a temperature from about 2° C. to about 25° C. for up to a week after the first and the second composition are combined.

18. A method of treatment of a condition selected from gastritis, gastroesophageal reflux disease, dyspepsia, peptic ulcer disease, laryngopharyngeal reflux, gastric and duodenum ulceration and Zollinger-Ellison syndrome, said method comprising administering to a patient in need thereof a pharmaceutical formulation comprising
a first composition being a non-aqueous liquid comprising a substituted benzimidazole selected from omeprazole, esomeprazole, and pharmaceutically acceptable salts thereof as a solution in dimethyl sulfoxide; and
a second composition being an aqueous liquid comprising a base;
wherein the first and the second compositions are configured to be combined before use to provide a combined liquid medicament having a pH in the range of between 7.5 to 9.5.

19. A kit comprising a first compartment containing a first composition as defined in claim 1;
a second compartment containing a second composition as defined in claim 1; and
wherein the first and second containers are adapted to allow fluid communication between them on actuation to form a combined liquid medicament as defined in claim 1.

20. A kit as defined in claim 19 wherein the first composition is present in an amount of from 1 to 5 mL, preferably about 2 mL.

21. A kit as defined in claim 19 wherein the second composition is present in an amount of from 25 to 100 mL, preferably about 68 mL.
